(19) United States
(12) Patent Application Publication
(10) Pub. No.: US 2012/0186999 A1
(43) Pub. Date: Jul. 26, 2012

(54) ELECTROCHEMICAL SENSOR

(75) Inventors: David John Walton, Coventry (GB); Xiaobo Ji, Changsha City (CN); Craig Edward Banks, Sale (GB)

(73) Assignee: KANICHI RESEARCH SERVICES LIMITED, Coventry (GB)

(21) Appl. No.: 13/266,442

(22) PCT Filed: Apr. 27, 2010
(86) PCT No.: PCT/GB2010/000829

§ 371 Date: Mar. 30, 2012

(30) Foreign Application Priority Data
Apr. 27, 2009 (GB) 0907195.2

(51) Int. Cl. G01N 27/30; G01N 33/50; B82Y 30/00

(52) U.S. Cl. 205/780.5; 204/400; 204/412; 204/435; 204/431; 204/415; 205/787; 205/775; 205/782; 977/773

(57) ABSTRACT

The invention provides an electrochemical sensor comprising an electrode assembly which comprises at least two electrodes, one of the electrodes comprising a metal species capable of catalysing the oxidation of hydrogen and/or methane. The sensor may be used in the detection and quantification of hydrogen and/or methane in exhaled breath, for example as a means of diagnosing lactose malabsorption or lactose intolerance.

The present invention relates to an electrochemical sensor and its use in the diagnosis of digestive disorders. In particular, the invention relates to the use of an electrochemical sensor in the detection and quantification of components such as hydrogen and/or methane in the exhaled breath of a subject, e.g. as a means of diagnosing lactose malabsorption or lactose intolerance.

In many circumstances it is desirable to test an individual's health for digestive disorders. For example, a large proportion of the human population loses the ability to digest and absorb lactose due to the decrease in activity of the enzyme lactase phlorizin hydrolase in the small intestine. Lactose malabsorption may be asymptomatic or induce symptoms similar to those of functional bowel disorders and irritable bowel syndrome, including gaseousness, flatulence, bloating, diarrhoea and abdominal pain. The term ‘lactose intolerance’ is generally considered to refer to a condition in which abdominal symptoms are experienced after the ingestion of lactose, usually in milk or dairy food.

Recent information suggests that the global population of lactose intolerance sufferers is substantial, and growing. For example, approximately 16% of Americans are lactose intolerant and, in Europe, 50% of adult Italians cannot digest and absorb lactose normally (Argnani et al., World J. Gastroenterol. 14(40): 6204-6207, 2008). Beyerlein et al. (Aliment. Pharmacol. Ther. 27: 659-665, 2008) performed a lactose breath test on 1127 patients in Zurich between July 1999 and December 2005 with 376 patients (˜33%) producing a positive test. In addition they performed the test on non-Swiss individuals which indicated 54% of the patients were lactose intolerant.

Currently, lactose intolerance can be determined using two main methods. The first of these is the “Lactose Tolerance Test” where blood is taken from a patient and the glucose level tested. A drink of lactose is given and, over the next 2-3 hours, blood samples are taken to see how much glucose is in the blood; this is used to determine the extent to which lactase is present in the digestive system (note that this test is not used on young children, but rather an “Acid Stool Test” is performed).

The second test is the “Hydrogen Breath Test” in which the concentration of hydrogen in exhaled breath is determined following ingestion of lactose. This test exploits the fact that normal colonic flora metabolise lactose into hydrogen and short chain fatty acids. The hydrogen is absorbed from the intestines, carried through the blood stream to the lungs and exhaled. Beforehand, the patient must fast for 12 hours, then they are required to drink a solution containing lactose (usually 25 grams). A sample of breath is then collected every 15 minutes over a 2 hour period. The amount of hydrogen in the breath is monitored as the solution is digested. An increase in hydrogen in breath of 20 ppm (from the baseline as measured before the lactose test) indicates that the subject has improper digestion of lactose and so is medically recognised as being ‘lactose intolerant’.

The advantages of the Hydrogen Breath Test are that it is non-invasive and simple to perform. It may be performed at home by the patient who is able to collect breath samples which are then sent away for analysis, or in a clinical setting administrated by a medical practitioner in which case the exhaled gas can be analysed directly on-site. Typically, samples of breath are analysed using chromatography. In the USA, in clinical settings, breath samples can be analysed on site using a ‘Quintron MicroLyzer’. Some, however, prefer to use the more portable system, the ‘micro H2’ which uses fuel cell technology. For both of these devices, however, constant calibration is required to establish a suitable base-line, which adds to the overall cost and time required to perform the measurement. Calibration with hydrogen gas (and other carrier gases) also has health and safety implications. Not only is the cost of such systems relatively expensive per sample, but the upkeep and maintenance of the machines is also costly.

Additionally, certain compounds which are present in breath, such as sulphur compounds and the like, act as catalyst poisoning compounds which can bind detrimentally to the fuel cell technology in the ‘micro H2’ device hereby increasing the time the sensor needs to overcome this (i.e. to re-equilibrate or re-set back to zero).

When diagnosing lactose intolerance, there is a further disadvantage in using current devices which only measure expired hydrogen concentrations. Hydrogen non-excretion (i.e. a false negative lactose hydrogen breath test) occurs in up to 20% of patients with lactose malabsorption. This is due to methane-producing bacteria in the bowel that use hydrogen to reduce carbon dioxide to methane, or may also occur as a result of prior administration of antibiotics (which influence the type and quantity of colonic bacteria).

The applicants have appreciated that there is a need for alternative methods for the detection and quantification of hydrogen and/or methane in exhaled breath, e.g. in the diagnosis of digestive disorders such as lactose intolerance. In particular, there is a need for such methods which are cost-effective and which can provide instantaneous results, i.e. which do not require samples to be sent away for laboratory analysis. Methods which enable patients to carry out home testing are particularly desirable to the extent that these enable patients to manage their condition.

A novel electrochemical sensor has now been developed which meets these needs and which may be used in the detection of various components in exhaled breath, in particular in detecting gases which are susceptible to electrochemical oxidation, such as hydrogen and/or methane.

In one aspect the invention thus provides an electrochemical sensor comprising an electrode assembly which comprises at least two electrodes, one of said electrodes comprising a metal species capable of catalysing the oxidation of hydrogen and/or methane.

Specifically, the sensor comprises an electrode assembly which is cheap to produce and which can therefore be discarded after a single use. Since each electrode assembly is disposable, catalyst poisoning compounds likely to be found in breath are not a problem and no re-equilibration is required. The sensor also has the advantage over conventional sensors that constant calibration is not required, thereby further reducing the cost involved in performing the measurement.

The sensor according to the invention includes an electrode assembly which comprises a plurality of electrodes. Typically this will comprise at least two electrodes, i.e. a working electrode adapted to detect one or more (preferably one) target species in the sample of gas and a combination counter/reference electrode, both disposed in an electrolyte which permits electrical contact of the sample with the electrodes. However, preferably, at least three electrodes are provided in the sensor, i.e. a working electrode, counter electrode and a reference electrode. The use of a separate reference electrode or separate reference material (i.e. when using a combination counter/reference electrode) provides for a calibration-less sensor.

Any material which supports the passage of charge (i.e. electrons) may be used to form the electrodes for use in the electrode assembly herein described. Such materials include both conducting and semi-conducting materials.

In certain embodiments of the invention, more than one target species may be detected simultaneously using the sensor. For example, the sensor may be adapted to detect hydrogen and methane simultaneously; as previously noted, this is particularly beneficial in the context of diagnosing lactose intolerance. Simultaneous detection of more than one species may be achieved by providing more than one working electrode in which the different working electrodes are adapted to detect different target species. Where more than one working electrode is present, these may each have their own corresponding counter and reference electrodes. However, for reasons of simplicity, it is envisaged that these will share a common counter and reference electrode. Alternatively, a single working electrode may be used to detect multiple target species. This may, for example, comprise a plurality of functional components capable of catalysing the oxidation of a target gas in which the functional components are independently responsive to the different target species. In the case where the different target species undergo electrochemical oxidation at different oxidation potentials, a single working electrode comprising a single functional component which catalyses the oxidation of each target species may alternatively be used.

The electrodes will generally be provided on a suitable solid support or substrate and may be produced, for example, by thick film deposition of a suitable ink material. Suitable support materials include any inert, non-conducting material such as glass, plastic or ceramic. Preferred substances are high-dielectric polymeric materials such a polyvinyl chloride, polyester or polycarbonate.

The working electrode (or electrodes) will comprise a material (also referred to herein as a ‘functional component’) which is capable of electro-chemically oxidising the target gas, e.g. hydrogen. For example, this may comprise a material having catalytic activity for the oxidation of hydrogen or methane, or both hydrogen and methane gases. Suitable metals which may be used to form the working electrode include various transition metals and their oxides, for example platinum, palladium, gadolinium, copper, etc. or oxides thereof. A particularly preferred material for use in forming the working electrode is platinum which catalyses the dissociation of dihydrogen to hydrogen ions.

When detecting hydrogen the method is based on the catalytic oxidation of hydrogen at the anode and the simultaneous reduction of oxygen at the cathode which produces a flux of electrons, i.e. an electric current between the electrodes. The electric current ceases when the oxidisable component (e.g. hydrogen) in the volume of gas delivered to the anode is completely oxidised. The amount of current generated is directly proportional to the amount of the oxidisable component, i.e. the target species. Where the target species is hydrogen, the electrode reaction which takes place at the anode is as follows:

2H₂→4H⁺+4e⁻

Although the working electrode may be a macroelectrode, it is preferable to use materials which serve to increase the surface area of the electrode in order to provide a higher and more rapid response (the magnitude of the electrochemical signal is largely dependent on the surface area of the electrode which is directly exposed to and in contact with the sample of gas). Electrode materials having a nano or micron-sized metal surface are thus preferred for use in the invention. These may comprise nano- or micron-sized particulates either bound to, or otherwise immobilised on, the surface of the electrode material. The nano- or micron-sized particles may, for example, be chemically bound (e.g. covalently linked) to the electrode surface through the use of known chemical linkers. Alternatively, such materials may simply be immobilised on the surface of the electrode following solvent evaporation during a thick film deposition process. For example, these may be provided in the form of a layer on a screen printed carbon electrode layer.

Particulate materials for use in the invention may comprise particles up to 1000 μm in size, preferably 1 to 100 μm in size. However, from the point of view of improved signal-to-noise ratio, nanoparticulate materials are preferred. Additionally the cost of using a random dispersion of metal nanoparticles compared to that of a macro- or micro-electrode of the metal (especially platinum) is evident. Suitable nanoparticulate materials are those having particle sizes in the range 1 to 100 nm, more preferably about 1 to 50 nm. These sizes allow a greater contribution from surface phenomena, e.g. catalytic sites and crystal facets at the surface of the particles.

The particulate materials for use in the invention may be chemically synthesised using methods known to those skilled in the art, for example, by chemical solution methods.

Where particulate (e.g. nanoparticulate) materials are used, these may be bound in any convenient form, for example in the form of a sheet or fibre, which may be coated or printed onto a suitable electrode support. For example, these may be embedded within a sheet or fibres of electrically conductive material. Alternatively, these may be provided as a compact particulate mixture. The use of printed or compacted particulate material, such as nanometre to micrometre sized metals, is especially preferred for ease of construction of the sensor.

Metal decorated nanomaterials (e.g. nano platinum on micron sized palladium) may also be used. One preferred embodiment is screen printed electrodes which are decorated with desired nano or micron sized particles.

The working electrode will generally be used in conjunction with counter and/or reference electrodes deposited on the same substrate. The selection of materials for the counter and/or reference electrode may readily be determined by those skilled in the art. Carbon, metal or metal decorated carbon are particularly suitable materials for use as counter electrodes. Suitable reference electrode materials include silver chloride and mercury-based materials such as mercury chloride and mercury sulphide. The use of a silver/silver chloride reference/counter electrode is particularly preferred.

The use of a reference electrode or reference material enables a calibration-less system to be provided. Well known reference electrodes which may be used include silver-silver chloride electrodes. The reference electrode or reference material may comprise a compound (either bound or otherwise immobilised on the electrode surface) which is not sensitive to the target species to be detected (e.g. hydrogen) but yet provides a redox useful signal from which to quantify the signal from the working electrode. Electron transfer agents are particularly suitable for this purpose and may be derived from ferrocene compounds (e.g. decamethylferrocene or poly(vinylferrocene)), quinone compounds (e.g. napthaquinone) or other redox compounds such as anthracenes (e.g. vinylanthracene). Such compounds may be substituted by electron withdrawing or donating groups as required to alter their electron transfer properties thereby ensuring that the electrochemical signals for the two compounds are appropriately spaced apart.

Typically the electrodes will be provided in the form of screen printed electrode materials. The screen printing (also known as thick film printing) of electrochemical sensing platforms is attractive due to their reproducibility, simplicity and ability to produce large volumes at a low production cost allowing large volumes of sensors to be fabricated. Screen printing involves the use of a thixotropic fluid which is spread evenly across a mesh screen which defines the shape and size of the desired electrode. This thixotropic fluid contains the functional component of the electrode (e.g. a nanoparticulate powder of one of the metals mentioned above), a binder, a vehicle (e.g. a solvent) and one or more modifiers. For example, it may also contain graphite, carbon black, solvents and a polymeric binder. The binder serves to hold the components onto the substrate and merges with the substrate during subsequent high temperature firing. The vehicle acts as a carrier for the active component and typically comprises a volatile solvent which evaporates during firing. Once the mesh screen is peeled away, a well defined electrode pattern is provided on the substrate material. Firing at high temperature in a carefully controlled atmosphere (e.g. this should be free from particulates, water vapour, etc.) completes the production process.

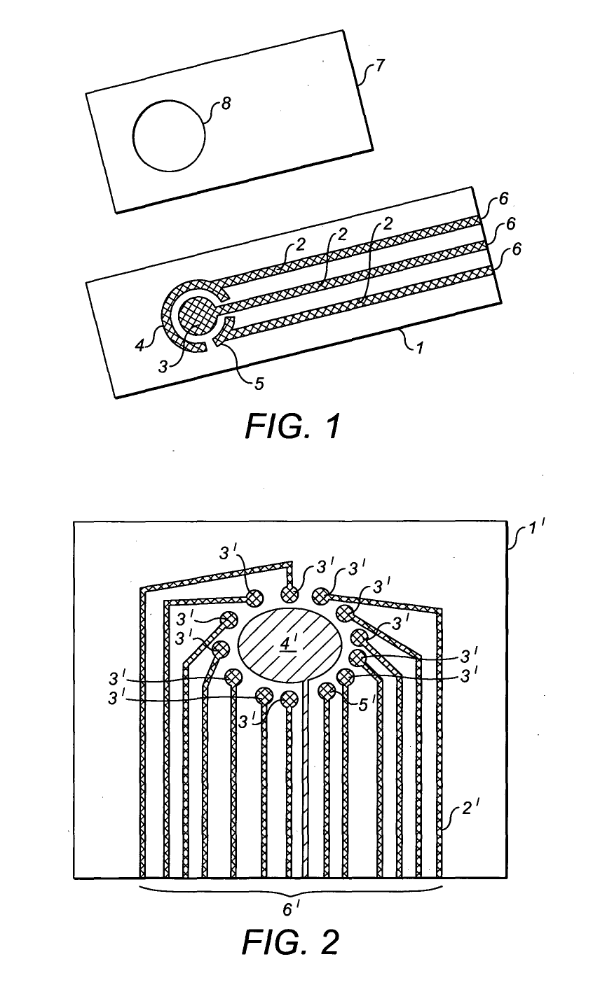

Suitable screen printed electrode materials may take any shape or configuration. For example these may be provided in a so-called Shepherd's Crook design as illustrated in accompanying FIG. 1. Alternatively, a plurality of working electrodes may be employed in combination with a common reference electrode and common counter electrode in a set-up as shown in FIG. 2. Other configurations are well known to those of skill in the art and are feasible. In order to improve the effectiveness of the sensor, the gap between the working and counter electrodes and any reference electrode and the counter electrode, should be kept to a minimum.

Other methods known to those of skill in the art may also be used for the production of the electrodes herein described, including photolithography, spin/sputter coating, electrochemical plating, vapour deposition, spray coating, ink jet printing, laser jet printing, roller coating, vacuum deposition, etc.

Typically, each electrode will comprise a single layer of the desired electrode material. However, multi-layered electrodes may also be employed in which each layer is applied to the substrate material using the same or different coating techniques. For example, the working electrode may comprise a layer of graphite particles coated with particles of an electrocatalytic metal such as platinum. Such particles may be held in place by a polymeric binder or otherwise linked to the surface of the graphite layer using known chemical linking agents. Alternatively, the nanoparticles may be dispersed in a coating on the electrode.

In the case where the electrocatalytically active particles are linked to the surface of the electrode, these may be linked using a variety of techniques known to those skilled in the art. For example, azide-functionalised gold nanoparticles may be prepared by treating standard, citrate-stabilised gold with appropriate ligands to yield thiolate-capped azide-functionalised particles which can then be attached via click chemistry.

Those portions of the electrodes which are not intended to come into contact with the gaseous stream may be provided with an inert coating in order to improve the electrical insulation of the electrodes. This coating will generally comprise an insulating dielectric layer which leaves exposed only the active portions of the electrodes.

Suitable electrolytes are well known and described in the literature. Where the electrolyte is a liquid, this may be aqueous or non-aqueous.

If desired, a liquid electrolyte may be contained within a suitable matrix, such as a thin strip of absorbent material, e.g. a gauze or mesh. Otherwise this may be provided in the form of a “free” electrolyte. Where the electrode materials themselves comprise a mesh or matrix of fibres (e.g. electrospun fibres), or otherwise comprise a suitable mesh or matrix-like layer or coating, these may aid in maintaining the electrolyte in contact with the active portion of the electrodes. Where a layer or coating on top of the electrode material is provided in the form of a suitable mesh or matrix capable of retaining the electrolyte, this layer of coating is non-electroactive.

Ionic liquids may also be used as electrolyte materials and for this purpose may be provided within a suitable support material which serves to retain the liquid in contact with the electrodes. Suitable support materials include inert materials which do not interact with the ionic liquid or the components of the gaseous stream. Such materials are either sufficiently porous to retain the liquid or can be granulated to provide a material with suitable pores. Zeolites and clays are one example of commercially available materials for this purpose. Other support materials include metal oxides such as titanium oxide, aluminum oxide, zirconium oxide, silicon dioxide and mixtures thereof such as silica-alumina. A particularly preferred support material is titanium dioxide.

Ionic liquids contain essentially only ions and are salts with relatively low melting points, e.g. below 100° C. Any suitable ionic liquid or mixture of ionic liquids may be used in the sensor of the invention. Suitable room temperature ionic liquids consist of bulky organic cations such as 1-alkyl-3-methylimidazolium, 1-alkylpyridinium, N-methyl-N-alkylpyrrolidinium, ammonium cations (e.g. tetraalkylammonium) and phosphonium cations (e.g. tetraalkylphosphonium). A wide range of anions may be employed from simple halide ions (e.g. F, Cl, Br) to inorganic anions such as tetrafluoroborate and hexafluorophosphate and to larger organic anions such as triflate or tosylate. Ionic liquids comprising a 1-alkyl-3-methylimidazolium cation are preferred in which the alkyl group is preferably C₁₋₁₀ alkyl, more preferably C₁₋₆ alkyl, e.g. C₁₋₄ alkyl. 1-butyl-3-methylimidazolium (bmim) is a particularly preferred cation which may be used in the form of the tetrafluoroborate salt, [bmim][BF₄], or the hexafluorophosphate salt, [bmim][PF₆].

An advantage of using ionic liquids is that these can be applied to the electrodes in a very thin layer or coating (e.g. of the order of nanometres) which provides a sensor with a fast response time (the thickness of the layer determines the rate at which the gaseous stream comes into contact with the working electrode).

In some cases, a solid electrolyte precursor may be provided which contacts the electrodes. On contact with the gaseous stream of exhaled breath, the water vapour present in the stream serves to hydrate the precursor to form an electrolyte. Examples of such materials include water-absorbing polymers such as super absorbent polyacrylate polymers (SAPs) which are cross-linked. One example of such a polymer material is sodium polyacrylate. Suitable electrolyte precursors also include gels or gel-like materials.

A semi-permeable membrane may be employed within the sensor to screen out cationic and/or anionic species which may be encountered in the sample and thus prevent these from penetrating to the working electrode. This membrane will typically comprise corresponding anionic and/or cationic groups capable of binding to such undesired species and can be specifically tailored to screen out unwanted gases such as sulphur compounds. The semi-permeable membrane may, for example, be cellulose acetate or a conventional dialysis membrane. Other suitable membrane materials include nafion, polyvinylsulphonate, carboxymethylcellulose, diethylaminoethylcellulose, polylysine and sulphonated polymers.

The sensor will generally comprise a protective chamber in which the electrode assembly is provided. Where the electrolyte is provided in the form of a liquid, this chamber will also serve to retain this liquid without leakage, i.e. it will provide a suitable seal. The precise shape and dimensions of the chamber are not critical to the operation or performance of the sensor provided that this is of sufficient capacity to enable the gas to be sampled by the electrode assembly. Generally speaking, chambers which have a smaller volume are preferred in that these serve to maximise uptake of hydrogen.

The sensor may further comprise a conduit or tube which directs the stream of gas directly onto the electrode assembly. This conduit may comprise a suitable mouthpiece into which the patient may exhale or, alternatively, suitable means for attachment of a bag or balloon carrying a gas sample collected off-site. The chamber and conduit will generally be formed from an inert, non-conductive material, such as plastic. Where a semi-permeable membrane is present, this will generally be disposed between the chamber which houses the electrode assembly and the working electrode.

In use, the sensor in accordance with the invention is connected to a reader which is configured to process electrical signals from the electrode assembly which are representative of the current. The current which passes between the working and counter electrodes is recorded and used to determine the concentration of the target species in the gaseous stream.

It is envisaged that the sensor herein described will typically be used just once (i.e. single-use) and that, following removal from the reader, it will be disposed of. In this regard, the assembly may include a fuse component containing a material which degrades under conditions of controlled passage of charge whereby to break the electrical contact and prevent re-use of the assembly. Suitable materials for use as the fuse component include conducting polymers such as polypyrroles, polythiophenes, polyanilines, polyazulenes, polyfurans, and the like, in combination with a requisite anion or mixture of anions such as perchlorate and the like. This component may also contain binders and other conventional polymer species, or other organic or inorganic compounds to enhance performance. Powders and particulates having particle sizes down to nanoscale are particularly suitable in order to provide the desired characteristics for printing or other means of production. Thin films of such materials may also be directly laid down.

The electrode assembly as defined herein forms a further aspect of the invention, as do the individual electrodes.

Methods of detecting the presence of, measuring the amount of or monitoring the levels of one or more desired components (e.g. hydrogen and/or methane) in a gaseous stream (e.g. exhaled breath) using the electrode assembly or electrochemical sensor as described herein form a further aspect of the invention.

In a further aspect the invention thus provides a method of detecting hydrogen and/or methane in exhaled breath which comprises the use of an electrochemical sensor as herein described. Methods of detecting hydrogen in the presence of methane and vice versa using the sensor are also provided.

Such methods comprise exposing the active portion of the electrode assembly to the target gas (e.g. hydrogen and/or methane) and measuring the current generated between the electrodes; the current generated is proportional to the mass of the gas present.

The sensor herein described may be used to detect the presence and concentration of various gases, in particular gases which undergo electrochemical oxidation at the working electrode of the sensor. However, preferred target species are hydrogen and/or methane.

The invention thus extends to the use of the sensor herein described for the electrochemical testing of exhaled breath.

The sensor is particularly suitable for the diagnosis of lactose intolerance. For such purposes, the patient is typically fasted (e.g. for a minimum of 8 hours) and then provided with a lactose containing drink (containing a dosage of 1 g of lactose per kg bodyweight). After a period of 2-3 hours the patient is then required to exhale into a sensor as herein described. Using such a procedure, an increase in hydrogen in breath of 20 ppm (from the baseline as measured before the test) indicates that the patient has improper digestion of lactose and so is considered ‘lactose intolerant’. More specifically, increased levels after ingestion of lactose for hydrogen values of 20-40 ppm indicate mild intolerance, 40-80 ppm indicates moderate intolerance, while above 80 ppm is severe lactose intolerance. As previously noted, in addition to a rise in hydrogen levels, a small proportion of sufferers also produce methane. A level of 12 ppm of methane in breath is similarly considered ‘lactose intolerant’.

Although the invention has been described primarily in relation to the detection and quantification of hydrogen and/or methane in breath for the purposes of diagnosing lactose intolerance, the methods and devices herein described also find use in diagnosing other digestive disorders. This may be done simply by changing the specific substrate (carbohydrate) from lactose. Table 1 provides details of appropriate test protocols for determination of lactose intolerance and intolerance to other carbohydrates, i.e. fructose, sucrose, d-xylose, sorbitol and lactulose.

TABLE 1

| Carbohydrate | Dosage | Positive result |
|---|---|---|
| Lactose | 1 g/kg in 250 mL H₂O | H₂ level increase > 20 |
| | | ppm over baseline. |
| | | CH₄ > 12 ppm over |
| | | baseline. |
| | | Combined increase (H₂ + |
| | | CH₄) > 15 ppm over |
| | | basline within test period |
| Fructose | 1 g/kg in 250 mL H₂O | Levels of H₂ > 20 ppm |
| Sucrose | 2 g/kg in 250 mL H₂O | H₂ level increases > 20 |
| | | ppm over baseline. |
| | | CH₄ > 15 ppm. |
| | | Combined increase (H₂ + |
| | | CH₄) > 15 ppm within test |
| | | period |
| d-Xylose | 1 g/kg in 250 mL H₂O | Increase of H₂ and CH₄ |
| | | indicates bacterial |
| | | overgrowth. |
| Sorbitol | 0.25-0.5 g/kg in | H₂ level increase is |
| | 250 mL H₂O | normal. |
| | | An increase of H₂ > 30 |
| | | ppm indicates sensitivity |
| | | to sorbitol |
| Lactulose | 10 g/kg in 250 mL H₂O | Two peaks; first increase |
| | | of 12 ppm or greater |
| | | followed by a second |
| | | much larger increase |
| | | after 1 hr. |
| | | Indicates bacterial |
| | | overgrowth |

Whilst the invention has been described primarily with respect to the detection and quantification of hydrogen and/or methane in exhaled breath, the methods herein described also find use in the detection and analysis of other gaseous components of breath. In particular, it is envisaged that these may be used in the so-called “urea breath test” which involves the electrochemical detection of ammonia in breath as a means of diagnosing the presence of the bacterium Helicobacter pylori (H. pylori) in the stomach.

H. pylori is responsible for inflammation, ulcers, and atrophy of the stomach and is usually treated with a course of antibiotics. The urea breath test is used not only to diagnose the presence of H. pylori, but can also be used to demonstrate that the bacterium has successfully been eliminated by treatment with antibiotics.

Existing methodology for the detection of H. pylori involves a patient swallowing a capsule containing urea in which the carbon atoms are suitably labelled (these are isotopes of carbon, such as radioactive carbon-14 or non-radioactive carbon-13). Over the next 10-30 minutes, samples of exhaled breath are analysed for the presence of the isotope-labelled carbon dioxide indicating that the urea has been decomposed; this indicates that urease, and hence H. pylori, is present in the stomach (urease is an enzyme that catalyses the hydrolysis of urea into carbon dioxide and ammonia, i.e. (NH₂)₂CO+H₂O→CO₂+2NH₃). For the two different forms of urea, different instrumentation is required; carbon-14 is normally measured by scintillation, carbon-13 by isotope ratio mass spectrometry (IRMS). For analysis of carbon-13, a baseline sample before taking urea is required for comparison with a post-urea sample. The difference between the pre- and post-urea measurements is used to determine the extent of the H. pylori infection by comparison with a cut-off value; results below the cut-off value are considered to be negative, whereas those above are considered positive. The cut-off value itself is determined by comparing the results from patients with two or more different detection methods.

The methods herein described permit the detection and quantification of ammonia in the breath of patients with a high degree of sensitivity and without the need for constant calibration.

Viewed from a further aspect the invention thus provides an electrochemical sensor as herein defined wherein at least one of the electrodes comprises a material capable of the electrochemical oxidation of ammonia. An electrode assembly for use in such a sensor is also provided. Also provided are methods of detecting the presence of, measuring the amount of, or monitoring the level of ammonia in exhaled breath (e.g. for use in diagnosing the presence of H. pylori in the stomach of a patient) using such a sensor or electrode assembly.

In such methods, either direct or indirect oxidation of ammonia is performed at the working electrode. Specifically, the direct electrochemical oxidation of ammonia (at the anode) proceeds as follows:

4NH₃(g)−3e⁻→3NH₄⁺+½N₂(g)

On the reverse (reductive scan) the following process takes place:

NH₄⁺ [symbol] NH₃(g)+H⁺

in which a proton formed via dissociation of NH₄⁺ is electrochemically reduced to produce hydrogen:

H⁺+e⁻+½H₂(g)

Suitable working electrode materials for use in this aspect of the invention include carbon-based materials such as those comprising boron-doped diamond, glassy carbon, edge plane pyrolytic graphite, basal plane pyrolytic graphite, carbon nanotubes (including both multi-walled and single-walled tubes), glassy carbon spheres and boron-doped diamond particles. Of these, glassy carbon is particularly preferred. Those materials having a large proportion of edge plane-like sites or defects are also particularly preferred. Metal electrodes such as platinum, gold and silver, as well as metal oxides such as TiO₂, etc. may also be employed. Electrode materials comprising nanoparticulates of any of the above are highly desirable.

The electrolyte may comprise any of the materials described herein in relation to the detection of hydrogen and/or methane. Although these may include water-based electrolytes, it is particularly desirable that these should be non-aqueous in order to avoid drying out. Ionic liquids and non-aqueous solvents are particularly preferred. One such solvent is propylene carbonate optionally in combination with tetra butyl-ammonium perchlorate.

When using carbon-based electrodes, intercalation processes can in some cases effectively passivate the surface of the electrode so precluding any useful measurements. For example, intercalation processes may dominate when using basal plane pyrolytic graphite electrodes. This effect can nevertheless be circumvented by using multi-walled carbon nanotube modified electrodes where the unique rigid structural morphology prevents propylene carbonate and supporting electrolyte intercalation so allowing quantitative electroanalysis.

Although ammonia can be detected directly, it is also possible for this to be detected indirectly. This involves the monitoring of an electrochemically active species which chemically reacts with the target analyte ammonia giving rise to a variation in the electrochemical signal. For example, this may involve the reaction of ammonia and hydroquinone where ammonia reversibly removes one proton from the hydroquinone resulting in the observation of a new voltammetric wave. The current of this new wave can be used to quantify the amount of ammonia present. The mechanism for the electrochemical reaction of hydroquinone with ammonia may be described as follows:

QH₂+NH₃ [symbol] QH⁻+NH₄⁺

QH⁻−2e⁻ [symbol] QH⁺

Where QH₂ is hydroquinine, QH⁻ is the deprotonated hydroquinone and QH⁺ is the protonated p-benzoquinone.

Certain preferred embodiments of the invention will now be described, by way of example only, with reference to the accompanying figures in which:

FIG. 1 is an exploded view of an electrode assembly in accordance with an embodiment of the invention;

FIG. 2 shows an electrode assembly in accordance with an alternative embodiment of the invention;

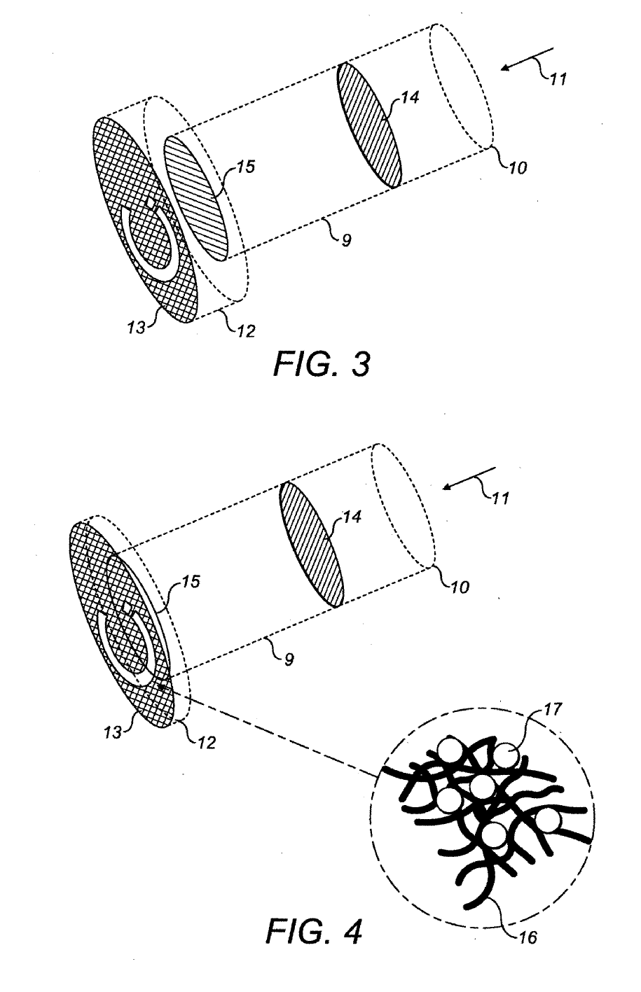

FIG. 3 shows an electrochemical sensor in accordance with an embodiment of the invention;

FIG. 4 shows an electrochemical sensor in accordance with an alternative embodiment of the invention;

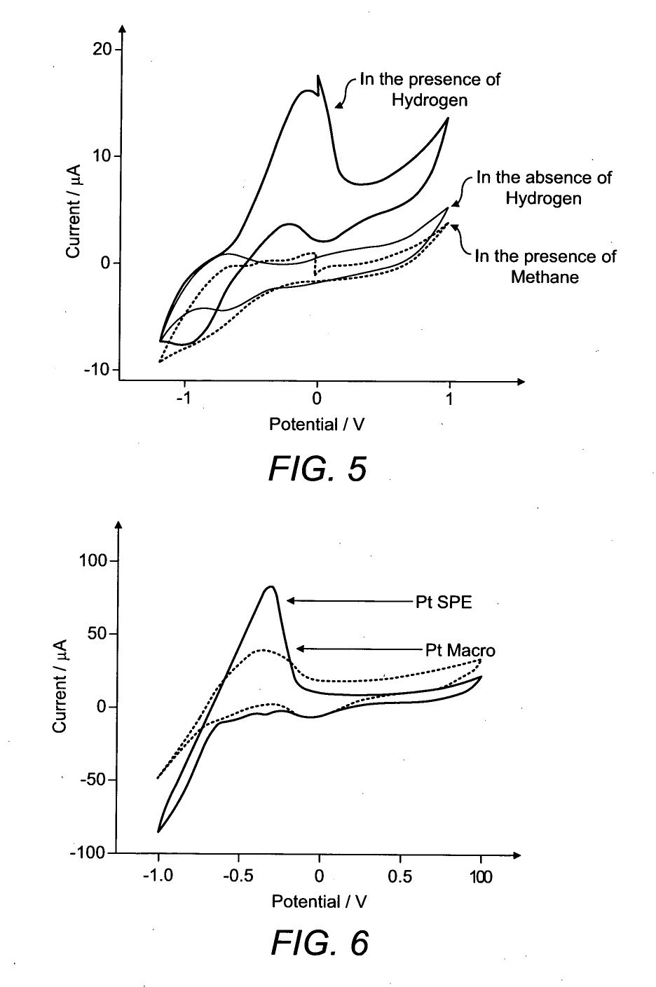

FIG. 5 shows the voltammetric profile for a Pt screen printed modified electrode in accordance with the invention; and

FIG. 6 shows the voltammetric profile for a Pt macroelectrode (Pt Macro) and a Pt screen printed electrode (Pt SPE) in accordance with the invention.

FIG. 1 shows an electrode assembly in accordance with an embodiment of the invention. A support 1, typically made of PVC, polycarbonate or polyester, supports three printed electrically conducting tracks 2 which define the working electrode 3, the counter electrode 4 and the reference electrode 5. The working electrode 3 provides a large surface area of exposed electrode with minimum separation between adjacent portions of the working and counter electrode 4. Reference electrode 5 is also provided in close proximity to the counter electrode 4. The configuration of electrodes shown is known as a ‘Shepherd's Crook’ electrode (but other configurations are feasible).

The electrical contacts 6 at the end of the tracks fit into a suitable reader (not shown). An insulating dielectric layer 7 is provided as a coating on top of the printed electrodes. This comprises an aperture 8 of a suitable size such that the active areas of the underlying electrodes remain exposed when in use. The aperture 8 may be formed by any suitable method. Metal underlayers (not shown) may also be provided to enhance electrode conductivity.

FIG. 2 shows an alternative electrode assembly in accordance with the invention in which support 1′ supports twelve working electrodes 3′ which share a common reference electrode 5′ and counter electrode 4′. Each electrode is provided with electrically conductive tracks 2′ having electrical contacts 6′ which enable connection to a suitable reader (not shown). When used in the detection of a single analyte, the plurality of working electrodes may be interrogated simultaneously to give an average reading (thereby improving the accuracy of the measurement) or may be individually addressed to give a similar analysis in sequential order. When used in detection of multiple analytes, each working electrode may be of differing composition (i.e. comprise different functional components) so as to each perform a different analysis. The support 1′ may be provided with a suitable covering layer (dielectric coating) as shown in FIG. 1.

FIG. 3 shows an electrochemical sensor in accordance with the invention. This may be used for analysing various components of exhaled breath, such as hydrogen, methane, etc. The sensor 9 comprises a conduit 10 through which a sample of breath 11 is passed. The conduit 10 is connected to a chamber 12 in which the electrode assembly 13 is housed. The base of chamber 12 is adapted to be docked with an electronic reader (not shown).

The sample of breath may be delivered from a bag in which a breath sample is stored following collection. However, typically the patient will breathe directly into the sensor 9 via the conduit 10. In this case, it would be expected that a mouthpiece (not shown) into which the patient may breathe would be provided at the end of the conduit 10. Within the conduit 10, a membrane 14 is provided in order to adjust the flow rate of exhaled air. A one-way valve 15 provided at the interface of the conduit 10 and chamber 12 ensures that there is no flow-back of air and improves monitoring of the desired components. The electrode assembly 13 is located within the chamber 12 in a liquid zone (electrolyte) and is positioned such that the stream of exhaled air impinges on the exposed portion of the electrode assembly.

FIG. 4 shows an alternative sensor in accordance with the invention in which components common to the sensor of FIG. 2 are identified by way of the same reference numerals. In this particular embodiment, the working electrode comprises a mesh of electro-spun fibres 16 in which the electrocatalytically active metal nanoparticles 17 are embedded. These fibres are capable of absorbing the liquid electrolyte material and enable the use of a chamber having a smaller volume.

The present invention is further illustrated by way of the following non-limiting examples:

### EXAMPLE 1

An electrode assembly is prepared as shown in FIG. 1. The working electrode is a platinum screen printed modified electrode comprising platinum nanoparticles immobilised on the surface of a carbon screen printed electrode. The counter and reference electrodes are formed from silver/silver chloride and are similarly formed by screen printing techniques. The electrodes are immersed in a liquid medium comprising distilled water to form a sensor.

The sensor is connected to a circuit and exposed to a gas stream comprising both hydrogen and methane gases. The output voltammogram for a single voltage scan of from −1 to 0 to +1 volts of the sensor is recorded at a scan rate of 100 mV/s and is shown in FIG. 5.

The platinum nanoparticles result in the selective electrochemical oxidation of hydrogen thereby producing a large and easily quantifiable peak which is proportional to the concentration of hydrogen. The electrochemical oxidation of methane occurs at a higher oxidation potential and thus does not detrimentally affect the electrochemical oxidation of hydrogen.

The voltammograms show the ability of the sensor to respond to changes in the concentration of hydrogen and methane in the gas stream. This may therefore be used to quantify the amount of both hydrogen and methane present in the sample.

### EXAMPLE 2

A sensor having the same composition as in Example 1 is prepared with the exception that the working electrode comprises a platinum macroelectrode (Pt Macro). The performance of this electrode is compared to that of the electrode of Example 1 having a platinum decorated screen printed electrode (Pt SPE).

FIG. 6 shows the voltammetric profiles obtained at both electrodes. Both are recorded at a scan rate of 100 mV/s and were recorded in distilled water versus a pseudo silver-silver chloride reference electrode.

Whilst a macro-electrode made from a similar metal may provide a useful signal at this concentration, the platinum nanoparticles provide a greater surface area and a modified proportion of active surface sites that may enhance selective reactivity. This is particularly useful at the lower gas concentrations which are encountered with the hydrogen breath test and in which the improved surface area allows an improved signal-to-noise ratio allowing a greater sensitivity.

1. An electrochemical sensor comprising an electrode assembly which comprises at least two electrodes, one of said electrodes comprising a metal species capable of catalysing the oxidation of hydrogen and/or methane.

2. A sensor as claimed in claim 1 which comprises a disposable electrode assembly.

3. A sensor as claimed in claim 1 wherein said electrode assembly comprises a working electrode adapted to detect one or more target species and a combination counter/reference electrode.

4. A sensor as claimed in claim 1 wherein said electrode assembly comprises a working electrode adapted to detect one or more target species, a counter electrode and a reference electrode.

5. A sensor as claimed in claim 1 which comprises a single working electrode adapted to detect multiple target species.

6. A sensor as claimed in claim 1 which comprises a plurality of working electrodes.

7. A sensor as claimed in claim 6 which comprises a plurality of working electrodes adapted to detect the same analyte.

8. A sensor as claimed in claim 6 which comprises a plurality of working electrodes adapted to detect a plurality of different analytes.

9. A sensor as claimed in claim 1 wherein the working electrode (or working electrodes) comprises a material capable of electrochemically oxidising the target analyte, e.g. hydrogen.

10. A sensor as claimed in claim 9 wherein said material is a transition metal or transition metal oxide, preferably platinum, palladium, gadolinium, copper or an oxide of any one of these metals.

11. A sensor as claimed in claim 1 wherein the working electrode, or in the case where a plurality of working electrodes is present, at least one of said working electrodes, comprises nano- or micron-sized metal particulates either bound to or otherwise immobilised on the surface of the electrode.

12. A sensor as claimed in claim 11 wherein the size of the particulates is up to 1000 μm, preferably 1 to 100 nm, e.g. 1 to 50 nm.

13. A sensor as claimed in claim 11 wherein said particulates are embedded within a sheet or fibres of electrically conductive material.

14. A sensor as claimed in claim 11 wherein said particulates comprise metal decorated nanomaterials.

15. A sensor as claimed in claim 1 which comprises a reference electrode or reference material comprising a compound adapted to provide a redox signal capable of quantifying the magnitude of the signal from the working electrode (or working electrodes) when the sensor is in use.

16. A sensor as claimed in claim 1 wherein the electrodes are provided in the form of screen printed electrode materials.

17. A sensor as claimed in claim 1 wherein said electrode assembly further comprises an electrolyte adapted to provide electrical contact of the analyte with the electrodes.

18. A sensor as claimed in claim 1 which comprises a protective chamber in which the electrode assembly is positioned and a conduit adapted to direct a gaseous sample directly onto the electrode assembly.

19. A sensor as claimed in claim 1 which further comprises a semipermeable membrane capable of preventing cationic and/or anionic species from penetrating to the working electrode or working electrodes.

20. A sensor as claimed in claim 1 which is adapted for the detection and/or quantification of hydrogen and/or methane in a gaseous stream (e.g. in exhaled breath).

21. An electrode assembly as defined in claim 1.

22. An assembly as claimed in claim 21 which is adapted for single use.

23. A method of detecting the presence of, measuring the amount of or monitoring the levels of one or more components (e.g. hydrogen and/or methane) in a gaseous stream (e.g. exhaled breath) using the electrochemical sensor of claim 1.

24. Use of an electrochemical sensor as claimed in claim 1 for the electrochemical testing of exhaled breath.

25. Use as claimed in claim 24 for the detection of hydrogen and/or methane in exhaled breath.

26. Use as claimed in claim 24 for the diagnosis of lactose malabsorption or lactose intolerance.

27. A reader adapted for use with an electrochemical sensor as claimed in claim 1.

28. An electrochemical sensor as claimed in claim 1 which is adapted for the detection of ammonia in a gaseous stream (e.g. in exhaled breath).

29. An electrode assembly for use in a sensor as claimed in claim 28.

30. A method of detecting the presence of, measuring the amount of, or monitoring the level of ammonia in exhaled breath (e.g. for use in diagnosing the presence of H. pylori in the stomach of a patient) using an electrochemical sensor as claimed in claim 28.
